(19) United States
(12) Patent Application Publication
(10) Pub. No.: US 2023/0108270 A1
(43) Pub. Date: Apr. 6, 2023

(54) STABILIZATION OF COMPOUNDS AS CYCLODEXTRIN COMPLEXES

(71) Applicant: University of Pittsburgh - Of the Commonwealth System of Higher Education, Pittsburgh, PA (US)

(72) Inventors: Bruce A. Freeman, Pittsburgh, PA (US); Francisco J. Schopfer, Pittsburgh, PA (US); Sonia R. Salvatore, Pittsburgh, PA (US)

(73) Assignee: University of Pittsburgh - Of the Commonwealth System of Higher Education, Pittsburgh, PA (US)

(21) Appl. No.: 17/909,601

(22) PCT Filed: Mar. 15, 2021
(86) PCT No.: PCT/US2021/022395

§ 371 Date: Sep. 6, 2022

Related U.S. Application Data
(60) Provisional application No. 62/992,036, filed on Mar. 19, 2020.

(51) Int. Cl. A61K 47/69; C07C 205/50; C08B 37/16

(52) U.S. Cl. CPC A61K 47/6951; C07C 205/50; C08B 37/0015

(57) ABSTRACT

A composition comprising a complex of a cyclodextrin with a nitroalkene.

This application claims the benefit of U.S. Provisional Application No. 62/992,036, filed Mar. 19, 2020, which is incorporated herein by reference.

### ACKNOWLEDGMENT OF GOVERNMENT SUPPORT

This invention was made with government support under Grant Nos. GM125944 and DK112854 awarded by the National Institutes of Health. The government has certain rights in the invention.

### BACKGROUND

Nitroalkene fatty acids (NO₂-FA) have been shown to play a protective role in numerous experimental settings that include endotoxin-induced vascular inflammation, endotoxemia and multi-organ injury, inflammatory bowel disease (IBD), allergic airway disease, tumor cell growth, invasion and metastasis, renal ischaemia and reperfusion (I/R) injury and diabetic and other forms of chronic kidney disease, pulmonary arterial hypertension (PAH), myocardial I/R injury, hypertension, and atherosclerosis.

10-nitro-octadec-9-enoic acid (NO₂-OA) requires storage at −80° C., and is labile at temperatures above −20° C., in the presence of water, upon exposure to atmospheric moisture, and/or in the presence of base, nucleophiles, nucleophilic amino acids, amines and proteins. This instability is a consequence of the reversible reaction with nucleophiles via a Michael addition reaction, a reaction catalyzed by a base. The reversible nature of this reaction results in the decomposition of the nitroalkene derivative through isomerization of the nitroalkene C—C double bond, double bond migration, dimerization reaction between two NO₂-OA molecules, and oxidation.

Conventionally, NO₂-OA is stabilized using oils as a way to reduce the impact of conditions known to cause its degradation (e.g., water content, base, nucleophiles, temperature). The conventional approaches used to date are all viscous liquid formulations that need to be maintained refrigerated, have limited shelf life, react with components of hard capsule surfaces, spilling has to be prevented, overall increasing the manufacturing, storage, distribution and overall clinical development costs. Oils that are used in nitroalkene fatty acid solvation included olive oil, sesame oil, and partially purified or synthetic oil preparations (synthetic triacylglycerols).

### SUMMARY

One embodiment disclosed herein is a composition comprising a complex of a cyclodextrin with a nitroalkene.

Another embodiment disclosed herein is a composition comprising a complex of a cyclodextrin with an active compound, wherein the active compound is:

a nitroalkene is a structure of formula I:

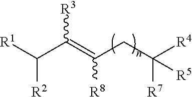

wherein R¹ is hydrogen, C₁-C₂₄ alkyl, C₁-C₂₄ alkenyl, or C₁-C₂₄ alkynyl;

R², R³, R², and R⁸ are each independently, hydrogen, oxygen, C₁-C₂₄ alkyl, NO₂, OH, or OOH;

R⁴ is a terminal COOR⁶ group, wherein R⁶ is hydrogen, or a C₁-C₂₄ alkyl;

R⁵ is hydrogen, C₁-C₂₄ alkyl, or R⁴ and R⁵ collectively form ═C(R⁹)(R¹⁰), wherein R⁹ comprises C₁-C₂₄ alkyl, C₁-C₂₄ alkenyl, or C₁-C₂₄ alkynyl, or wherein R⁹ is a terminal COOR⁶ group, and R¹⁰ is hydrogen, NO₂, OH, or OOH;

n is from 1 to 24; and

wherein the nitroalkene fatty acid includes at least one NO₂ group;

a nitroalkene is a structure of formula II:

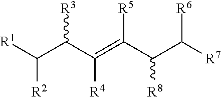

wherein R¹ is hydrogen, C₁-C₂₄ alkyl, C₁-C₂₄ alkenyl, or C₁-C₂₄ alkynyl;

R², R⁴, R⁵ and R⁶ are each hydrogen;

R² is a terminal COOR⁹ group, wherein R⁹ is hydrogen or a C₁-C₂₄ alkyl; and

R³ and R⁸ are each independently, hydrogen, oxygen, C₁-C₂₄ alkyl, NO₂, OH, ONO₂, NO, ONO or OOH, provided at least one of R³ or R⁸ is NO₂ and the other of R³ or R⁸ is hydrogen, ONO or ONO₂;

a nitro group-containing compound is a structure of formula III:

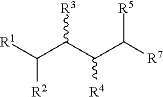

wherein R¹ is hydrogen, C₁-C₂₄ alkyl, C₁-C₂₄ alkenyl, or C₁-C₂₄ alkynyl;

R² and R⁵ are each hydrogen;

R⁷ is a terminal COOR⁶ group, wherein R⁶ is hydrogen or a C₁-C₂₄ alkyl; and
R³ and R⁴ are each independently, hydrogen, oxygen, C₁-C₂₄ alkyl, NO₂, OH, ONO₂, NO, ONO or OOH, provided at least one of R³ or R⁴ is NO₂ and the other of R³ or R⁴ is hydrogen, ONO or ONO₂;

a compound comprising a dicarboxylic acid of a structure of formula IV:

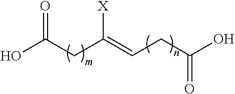

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂,

m is from 1 to 10; and

n is from 1 to 10;

a compound comprising a dicarboxylic acid of a structure of formula V:

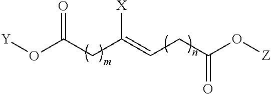

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;

Y and Z are each, independently, hydrogen or a C₁ to C₁₀ alkyl;

m is from 1 to 10; and

n is from 1 to 10; or

a compound comprising a dicarboxylic acid of a structure of formula VI:

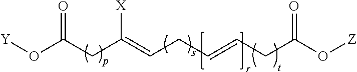

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;

Y and Z are each, independently, hydrogen or C₁ to C₁₀ alkyl;

p and t are each, independently, 1 to 10;

s is absent or 1 to 10, and

r is 1.

Further disclosed herein is a liquid composition comprising (a) water and (b) suspended or dissolved in the water, a solid powder comprising a complex of a cyclodextrin with a nitroalkene.

Another embodiment disclosed herein is a liquid composition comprising (a) water and (b) suspended or dissolved in the water, a solid powder comprising a complex of a cyclodextrin with an active compound, wherein the active compound is a structure of formulae I-VI.

Another embodiment disclosed herein is a pharmaceutical composition comprising the complex composition and at least one pharmaceutically acceptable excipient.

Another embodiment is a complex of a nitroalkene fatty acid and a cyclodextrin.

Another embodiment disclosed herein is method comprising contacting a nitroalkene with cyclodextrin under conditions resulting in forming a complex of the nitroalkene with the cyclodextrin.

Another embodiment disclosed herein is a method comprising contacting a cyclodextrin with an active compound under conditions resulting in forming a complex of the cyclodextrin with the active compound, wherein the active compound is a structure of formula I-VI.

Another embodiment disclosed herein is a method comprising mixing together (a) a liquid carrier and (b) a solid powder comprising a complex of a nitroalkene and a cyclodextrin.

Another embodiment disclosed herein is a method for treating a condition in a subject, comprising administering any of the compositions disclosed herein to a subject in need thereof, wherein the condition is an inflammatory condition, an immune disease, psoriasis, obesity, metabolic syndrome, acute kidney disease, chronic kidney disease, focal segmental glomerulosclerosis, atherogenesis, adipogenesis, neointimal proliferation, kidney I/R and xenobiotic injury, focal myocardial I/R injury, Ang II-induced systemic hypertension, pulmonary hypertension, cancer, cardiac and pulmonary fibrosis, liver fibrosis, non-alcoholic steatohepatitis (NASH), non-alcoholic fatty liver disease (NAFLD), breast cancer, ovarian cancer, inflammatory bowel disease, nociception, stroke, motor neuron degeneration, diabetes, aneurysm, aortic stiffness, lupus erythematosus, STING-associated vasculopathy with onset in infancy (SAVI), asthma, chronic obstructive pulmonary disease (COPD), or focal segmental glomerulosclerosis.

The foregoing will become more apparent from the following detailed description, which proceeds with reference to the accompanying figures.

### BRIEF DESCRIPTION OF THE DRAWINGS

FIG. 1 is a graph showing a comparison of yields from α-cyclodextrin and β-cyclodextrin inclusion complexes of the fatty acid nitroalkene 10-nitro-octadec-9-enoic acid (NO₂-OA). Area refers to the area established by the UV signal followed at 210 nm during the HPLC run. The area under the UV-HPLC trace corresponding to the fatty acid is quantified and represents the amount of fatty acid present in the sample. 10 ul aliquots were injected into the HPLC-UV and signal was followed using a diode array spectrophotometer between 190 and 700 nm. Ratio (lipid/cyclodextrin) refers to the molar proportion of fatty acid to cyclodextrin that was used during the preparation of the inclusion complex.

FIG. 2 is a graph showing the recovery percentages obtained from the process of making the inclusion complexes. All three attested ratios resulted in the efficient incorporation and stabilization of NO₂-OA in the inclusion complexes. For recovery calculations, inclusion complexes were extracted using methanol, and injected for evaluation by HPLC-UV (quantification, purity and integrity) and HPLC-MS/MS (integrity confirmation).

FIG. 3 is a table showing a stability evaluation scheme for a NO₂-OA/cyclodextrin complex powder disclosed herein. Exposure to a different temperature in the presence of air and the same humidity conditions were tested.

FIG. 4 are graphs showing the recovery of the NO₂-OA from the β-cyclodextrin inclusion complexes after exposure to the different conditions defined in FIG. 3. No significant changes were observed for NO₂-OA/β-cyclodextrin inclusion complex stability during the 28 day period. The stability of the sample subjected to 70° C. was only tested up to 14 days. Incubation of pure NO₂-OA or NO₂-OA stabilized in oils results in significant degradation under these conditions with the formation of dimers, double bond isomerization, double bond migration and oxidation products. These decomposition products were not observed in the β-cyclodextrin stabilized samples.

FIG. 5A shows the structure of the initial (E)10-NO₂-OA isomer (>99%, boxed structure) present in the testing material used to assess stability. Structures of previously identified and characterized (in triglyceride-based oil formulations) oxidation, isomerization and dimerization NO₂-OA decomposition products are shown as well as the categories to which they belong.

FIG. 5B is a chromatogram of a NO₂-OA/β-cyclodextrin complex disclosed herein evaluated 14 days after exposure to 55° C. under an air atmosphere. The main peak observed in the chromatogram corresponds to pure 10-NO₂-OA, with no apparent formation of oxidation or isomerization products.

Dimerization products were not observed and the peak observed in the 9 min RT area was present at similar intensities in blank injections.

FIG. 5C shows the overlayed chromatograms of NO₂-OA/β-cyclodextrin complex disclosed herein evaluated 14 days after exposure to 55° C. under an air atmosphere and a standard mixture containing 10-NO₂-8,9-alkene, (E)10-NO₂-OA and (Z)10-NO₂-OA. Overlayed chromatograms show absence of degradation products in the tested sample after 14 day exposure to 55° C.

FIG. 6A represents a graph demonstrating that the process is reproducible and results in full incorporation of 10-NO₂-OA into the NO₂-OA/β-cyclodextrin inclusion complexes. Independent batches were evaluated and quantified in triplicate.

FIG. 6B is a graph showing external standard curves used to quantify levels of NO₂-OA content in the β-cyclodextrin inclusion complexes. Quantification was performed by HPLC-UV using external standard curves using pure 10-NO₂-OA at different concentrations, which were injected into the HPLC-UV and areas under the curve quantified.

FIGS. 7A-7C shows graphs demonstrating the stability a NO₂-OA/β-cyclodextrin complex disclosed herein when dissolved in water. NO₂-OA associated but not contained in the NO₂-OA β-cyclodextrin inclusion complexes rapidly equilibrate after dissolution in water and decays during the first hour (FIG. 7A). After that, the concentration and integrity of the inclusion complex remains stable for the remaining of the tested time. The concentration on day 10 was re-evaluated as an indicator of stability (FIG. 7B). In contrast, the same molar amount of 10-NO₂-OA was added to water, resulting in a rapid loss of 10-NO₂-OA in solution (within 4 hrs) (FIG. 7C).

FIGS. 8A-8C shows that 10-NO₂-OA/β-cyclodextrin inclusion complexes can be used to administer NO₂-OA, an oily fatty acid, in drinking water. Two concentrations were tested in mice, 0.31 mg/ml and 1.95 mg/ml. Dissolution of 10-NO₂-OA/β-cyclodextrin inclusion complexes did not produce any changes in drinking habits nor noticeable taste aversion (followed as a change in daily water intake), as β-cyclodextrin was masking the 10-NO₂-OA flavor. The drinking of 10-NO₂-OA/β-cyclodextrin inclusion complexes water solutions resulted in bioavailable 10-NO₂-OA as evidenced by the detection of 10-NO₂-OA and its metabolites in urine (not shown) and feces (chromatograms of main metabolites in feces shown in FIG. 8A and specific beta oxidation products of NO₂-OA and NO₂-SA in FIGS. 8B and 8C respectively). FIG. 8B shows the formation of β-oxidation products of 10-NO₂-OA (10-NO₂-SA) (gray trace), dinor-NO₂-SA (green trace), tetranor-NO₂-SA (red trace) and hexanor-NO₂-SA (blue trace). FIG. 8C shows the formation of the reduced β-oxidation products of the 10-NO₂-OA) (gray trace), dinor-NO₂-OA (green trace), tetranor-NO₂-OA (red trace) and hexanor-NO₂-OA (blue trace).

FIG. 9 is a graph showing that NO₂-OA is absorbed and metabolized upon drinking water fortified with NO₂-OA using β-cyclodextrin stabilized inclusion complexes, as indicated by its detection in plasma. These graphs also indicate that the process of absorption proceeds through the same pathways previously determined for 10-NO₂-OA. This includes incorporation and biodistribution through plasma triglycerides.

This Figure shows a significant amount of 10-NO₂-OA and its main metabolite 10-NO₂-SA incorporated into triglycerides as evidenced by the increased observed upon hydrolysis. Free acid components were quantified using HPLC-MSMS using deuterated internal standards for species confirmation and quantification purposes.

FIG. 10 shows plasma metabolite profiles of mice administered 10-NO₂-OA/β-cyclodextrin inclusion complexes in the drinking water for 1 day. Two concentrations were tested, 0.31 mg/ml and 1.95 mg/ml, with the 10-NO₂-OA metabolic profile of the 0.31 mg/ml condition shown. Dissolution of 10-NO₂-OA/β-cyclodextrin inclusion complexes did not produce any changes in drinking habits nor noticeable taste aversion (followed as a change in daily water intake). The drinking of 10-NO₂-OA/β-cyclodextrin inclusion complexes water solutions resulted in bioavailable 10-NO₂-OA as evidenced by the detection of 10-NO₂-OA metabolites in plasma. Formation of β-oxidation products of the reduced 10-NO₂-OA (10-NO₂-SA) (light blue trace, 18:0), dinor-NO₂-SA (gray trace, 16:0), tetranor-NO₂-SA (green trace, 14:0) and hexanor-NO₂-SA (red trace, 12:0) (upper panels) and formation of beta-oxidation products of the 10-NO₂-OA (blue trace, 18:1), tetranor-NO₂-OA (green trace, 14:1) and hexanor-NO₂-OA (green trace, 12:1) (lower panels) is shown. Left, a representative chromatogram shows the profile of free NO₂-OA and its metabolites in plasma, and the right panel shows the chromatograms after hydrolysis of triglycerides using acid-based hydrolysis method.

FIG. 11 shows an analysis of NO₂-OA profile of mice feces as well as its main reported metabolites. NO₂-OA was stabilized as an inclusion complex with β-cyclodextrin as disclosed herein and delivered to mice in drinking water to obtain daily doses of 10 and 50 mg/kg. In this case a dose of 50 mg/kg is shown. Feces metabolite profile shows uptake of nitro oleic acid and extensive metabolism. It has been reported that a large amount of NO₂-oA is excreted through the feces as NO₂-oA and as partially metabolized material.

This further that stabilized inclusion complexes can be solvated and administered to reach central circulation and display a predicted metabolic profile both in urine and in feces. Upper panels shows β-oxidation of reduced metabolites while the lower panel shows β-oxidation of the parent compound.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for describing particular embodiments and examples only and is not intended to be limiting.

“Administration” as used herein is inclusive of administration by another person to the subject or self-administration by the subject.

“Alkenyl” refers to a cyclic, branched or straight chain group containing only carbon and hydrogen, and contains one or more double bonds that may or may not be conjugated. Alkenyl groups may be unsubstituted or substituted. “Lower alkenyl” groups contain one to six carbon atoms.

The term “alkyl” refers to a branched or unbranched saturated hydrocarbon group, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1- hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Alkynyl” refers to a cyclic, branched or straight chain group containing only carbon and hydrogen, and one or more triple bonds. Alkynyl groups may be unsubstituted or substituted.

The term “amine or amino” refers to an —NRpRq group wherein Rp and Rq each independently refer to a hydrogen, (C₁-C₈) alkyl, (C₁-C₈) haloalkyl, and (C₁-C₆) hydroxyalkyl group.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

As used herein, “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, OH, alkoxy, amino, CO₂H, CO₂alkyl, aryl, and heteroaryl. Exemplary aryl groups include but are not limited to phenyl, naphthyl, tetrahydronaphthyl, chlorophenyl, methylphenyl, methoxyphenyl, trifluoromethylphenyl, nitrophenyl, and 2,4-methoxychlorophenyl.

The term “haloalkyl,” refers to a C₁-C₈ alkyl group wherein one or more hydrogen atoms in the C₁-C₈ alkyl group is replaced with a halogen atom, which can be the same or different. Examples of haloalkyl groups include, but are not limited to, trifluoromethyl, 2,2,2-trifluoroethyl, 4-chlorobutyl, 3-bromopropyl, pentachloroethyl, and 1,1, 1-trifluoro-2-bromo-2-chloroethyl.

The term “halogen” and “halo” refers to —F, —Cl, —Br or —I.

The term “heteroatom” is meant to include oxygen (O), nitrogen (N), and sulfur (S). The term “heteroaryl” is employed here to refer to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and preferably one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “heterocycle” refers to a monocyclic, bicyclic, tricyclic, or polycyclic systems, which are either unsaturated or aromatic and which contains from 1 to 4 heteroatoms, independently selected from nitrogen, oxygen and sulfur, wherein the nitrogen and sulfur heteroatoms are optionally oxidized and the nitrogen heteroatom optionally quatemized, including bicyclic, and tricyclic ring systems. The heterocycle may be attached via any heteroatom or carbon atom. Heterocycles include heteroaryls as defined above. Representative examples of heterocycles include, but are not limited to, benzoxazolyl, benzisoxazolyl, benzthiazolyl, benzimidazolyl, isoindolyl, indazolyl, benzodiazolyl, benzotriazolyl, benzoxazolyl, benzisoxazolyl, purinyl, indolyl, isoquinolinyl, quinolinyl and quinazolinyl. A heterocycle group can be unsubstituted or optionally substituted with one or more substituents.

“Heterocycloalkyl” denotes to a monocyclic or bicyclic ring system containing one or two saturated or unsaturated rings and containing at least one nitrogen, oxygen, or sulfur atom in the ring. The term “cycloalkyl” refers to a monocyclic or bicyclic ring system containing one or two saturated or unsaturated rings. The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “oxo” refers to a ═O atom attached to a saturated or unsaturated (C₃-C₈) cyclic or a (C₁-C₈) acyclic moiety. The ═O atom can be attached to a carbon, sulfur, and nitrogen atom that is part of the cyclic or acyclic moiety.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition. In certain embodiments, treating a disease refers to inhibiting metastasis of the disease.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

The compounds of the invention can exist in various isomeric forms, including configurational, geometric, and conformational isomers, as well as existing in various tautomeric forms, particularly those that differ in the point of attachment of a hydrogen atom. The term “isomer” is intended to encompass all isomeric forms of a compound of this invention, including tautomeric forms of the compound.

Certain compounds described here may have asymmetric centers and therefore exist in different enantiomeric and diastereomeric forms. The compounds of the invention can be in the form of an optical isomer or a diastereomer. Accordingly, the invention encompasses compounds in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents. Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

The term “prodrug” denotes a derivative of a compound that can hydrolyze, oxidize, or otherwise react under biological conditions, in vitro or in vivo, to provide an active compound, particularly a compound of the invention. Examples of prodrugs include, but are not limited to, derivatives and metabolites of a compound of the invention that include biohydrolyzable groups such as biohydrolyzable thiol adducts, nitrate esters, amides, biohydrolyzable esters, biohydrolyzable carbamates, biohydrolyzable carbonates, biohydrolyzable ureides, and biohydrolyzable phosphate analogues (e.g., monophosphate, diphosphate or triphosphate). For instance, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by BURGER'S MEDICINAL CHEMISTRY AND DRUG DISCOVERY 6th ed. (Wiley, 2001) and DESIGN AND APPLICATION OF PRODRUGS (Harwood Academic Publishers Gmbh, 1985).

Disclosed herein are complexes of an active compound (e.g., a compound that includes an electron-withdrawing groups such as a nitroalkene) and a cyclodextrin. Although not bound by any theory, in aqueous solutions cyclodextrins form inclusion complexes with an active compound through a process in which the water molecules located in the central cavity are replaced by either the whole active compound molecule, or by some lipophilic portion of the active compound structure. The tridimensional structure of the cyclodextrin molecule provides a hydrophobic barrel that can bind and protect the active compound. Once included in the cyclodextrin cavity (i.e., the hydrophobic barrel), the drug molecules may be dissociated through complex dilution by replacement of the included drug by some other suitable molecule, and the drug may be transferred to the matrix for which it has the highest affinity. Importantly, since no covalent bonds are formed or broken during the drug cyclodextrin complex formation, the complexes are in dynamic equilibrium with free drug and cyclodextrin molecules (R. A. Rajewski and V. J. Stella, “Pharmaceutical applications of cyclodextrins. 2. In vivo drug delivery’. J. Pharm. Sci. 85(11), 1142-1169 (1996)).

Contacting the active compound with at least one cyclodextrin may include dissolving or suspending cyclodextrin in a solvent or mixture of solvents to form a first solution or suspension. Similarly, the active compound may be dissolved or suspended in the same or different solvent or mixture of solvents to form a second solution or suspension. The first solution or suspension may then be combined to form the present complex between active compound and the at least one cyclodextrin. The complex may then be separated from the solution and optionally purified, resulting in a complex of stabilized.

Contacting an active compound with at least one cyclodextrin may alternatively include dissolving or suspending at least one cyclodextrin in a solvent or mixture of solvents to form a solution or suspension, and then adding an active compound to the solution or suspension to form the present complex. Contacting an active compound with at least one cyclodextrin may be conducted by other methods. For example, a solvent may be utilized which will fully dissolve both the active compound and the cyclodextrin. In another embodiment, the cyclodextrin may be dissolved or suspended in a solvent or mixture of solvents and then placed on a rotovaporator. The active compound may then be sprayed directly into the solution or suspension, either as a neat form or as a solution or suspension of active compound in a solvent or mixture of solvents. The contacting may also be accomplished by use of a biphasic solvent system. For example, the active compound may be combined in separate, immiscible solvents (either as suspensions or in solution). The immiscible solvents may then be thoroughly mixed until a complex is formed. The complex may then be isolated via one of the isolation techniques discussed herein. It may be desirable to conduct the contact step in the absence of solvents. For example, in a spray drying technique, a mist of nitroalkene may be sprayed or misted on neat cyclodextrin to produce the present complex.

The cyclodextrin may be dissolved or suspended in a solvent selected from the group including weakly non-polar to polar solvents. Illustrative solvents for the cyclodextrin include water, methanol, ethanol, n-propanol, iso-propanol, n-butanol, sec-butanol, iso-butanol, tert-butanol, high molecular weight alcohols, dimethyl formamide, diethyl formamide, ethylene glycol, triethylene glycol, glycerin, polyethylene glycol, formamide, acetone, tetrahydrofuran, dioxane, methyl ethyl ketone, high molecular weight ketones, ethyl acetate, acetonitrile, N,N-dimethylacetimide, dimethylsulfoxide, carbon disulfide, hexane, hexane isomers, cyclohexane, heptane, heptane isomers, mineral oil, diethylether, methyl tert-butyl ether, methylene chloride, chloroform, carbon tetrachloride, benzene, nitrobenzene, toluene, and mixtures thereof. In certain embodiments, the cyclodextrin is dissolved in water.

The active compound may be dissolved or suspended in a solvent selected from the group including non-polar to weakly polar solvents. Illustrative solvents for the active compound include methanol, ethanol, n-propanol, iso-propanol, n-butanol, sec-butanol, iso-butanol, tert-butanol, pentanol, high molecular weight alcohols, dimethyl formamide, diethyl formamide, ethylene glycol, triethylene glycol, formic acid, acetic acid, formamide, acetone, tetrahydrofuran, dioxane, methyl ethyl ketone, high molecular weight ketones, ethyl acetate, acetonitrile, N,N-dimethylacetimide, dimethylsulfoxide, carbon disulfide, hexane, hexane isomers, cyclohexane, heptane, heptane isomers, mineral oil, diethylether, methyl tert-butyl ether, methylene chloride, chloroform, carbon tetrachloride, benzene, nitrobenzene, toluene, and mixtures thereof. In certain embodiments, a nitroalkene as the active compound is dissolved in ethanol.

The optional step of removing the complex from solution or suspension may be performed by separation techniques. Illustrative separation techniques include one or more of precipitation, filtration, evacuation, lyophilization, spray drying, and distillation.

The stabilized active compound/cyclodextrin complex may be stored as a solid at a convenient temperature (e.g., −80 to 30° C., more particularly 4 to 22° C.) for desired period of time. In certain embodiments, the time period may be at least 360 days, more particularly at least 90 days.

The stabilized active compound/cyclodextrin complex may be stored as a solid at a convenient temperature (e.g., −80 to 30° C., more particularly 4 to 22° C.) for desired period of time to then be re-dissolved using water to obtain a solution or a suspension to be used to administer the active compound. In certain embodiments, the stabilized active compound/cyclodextrin complex is in the form of a powder. In certain embodiments, the time period for the powder storage may be at least 360 days, more particularly at least 90 days and the time period for the powder storage may be at least 14 days, more particularly at least or 10 days.

In certain embodiments, the active compound is a nitroalkene that includes at least one carbon-carbon double bond and at least one nitro group. In certain embodiments, the nitroalkene is a nitroalkene fatty acid. Certain nitroalkene fatty acids are described, for example, in U.S. Pat. No. 7,776,916.

One illustrative embodiment of a nitroalkene is a structure of formula I:

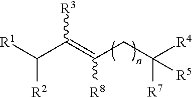

wherein R¹ is hydrogen, C₁-C₂₄ alkyl, C₁-C₂₄ alkenyl, or C₁-C₂₄ alkynyl;

R², R³, R⁷, and R⁸ are each independently, hydrogen, oxygen, C₁-C₂₄ alkyl, NO₂, OH, or OOH;

R⁴ is a terminal COOR⁶ group, wherein R⁶ is hydrogen, or a C₁-C₂₄ alkyl;

R⁵ is hydrogen, C₁-C₂₄ alkyl, or R⁴ and R⁵ collectively form) ═C(R⁹)(R¹⁰), wherein R⁹ comprises C₁-C₂₄ alkyl, C₁-C₂₄ alkenyl, or C₁-C₂₄ alkynyl, or wherein R⁹ is a terminal COOR⁶ group, and R¹⁰ is hydrogen, NO₂, OH, or OOH;

n is from 1 to 24; and

wherein the nitroalkene fatty acid includes at least one NO₂ group.

In certain embodiments of formula I, R¹ is C₁-C₂₄ alkyl, more particularly C₃-C₂₀ alkyl.

In certain embodiments of formula I, R² is hydrogen.

In certain embodiments of formula I, one of R³ or R⁸ is NO₂ and the other of R³ or R⁸ is hydrogen.

In certain embodiments of formula I, n is 3 to 20.

In certain embodiments of formula I, R⁴ is —COOH.

In certain embodiments of formula I, R⁵ is hydrogen.

In certain embodiments of formula I, R⁷ is hydrogen.

In certain embodiments of formula I, R⁴ is —COOH; R⁵ is methyl; and R⁷ is methyl.

In certain embodiments of formula I, R¹ is C₁-C₂₄ alkyl, more particularly C₃-C₂₀ alkyl; R² is hydrogen; one of R³ or R⁸ is NO₂ and the other of R³ or R⁸ is hydrogen.; R⁴ is —COOH; R⁵ is hydrogen; and R⁷ is hydrogen.

Another illustrative embodiment of a nitroalkene is a structure of formula II:

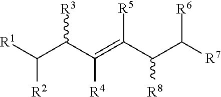

wherein R¹ is hydrogen, C₁-C₂₄ alkyl, C₁-C₂₄ alkenyl, or C₁-C₂₄ alkynyl;

R², R⁴, R⁵ and R⁶ are each hydrogen;

R⁷ is a terminal COOR⁹ group, wherein R⁹ is hydrogen or a C₁-C₂₄ alkyl; and

R³ and R⁸ are each independently, hydrogen, oxygen, C₁-C₂₄ alkyl, NO₂, OH, ONO₂, NO, ONO or OOH, provided at least one of R³ or R⁸ is NO₂ and the other of R³ or R⁸ is hydrogen, ONO or ONO₂.

In certain embodiments of formula II, R¹ is C₁-C₂₄ alkyl, more particularly C₃-C₂₀ alkyl;

R⁹ is hydrogen; and R³ is NO₂ and R⁸ is ONO₂ or R⁸ is NO₂ and R³ is ONO₂.

An additional illustrative embodiment of another nitro group-containing compound is a structure of formula III:

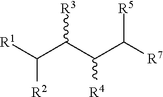

wherein R¹ is hydrogen, C₁-C₂₄ alkyl, C₁-C₂₄ alkenyl, or C₁-C₂₄ alkynyl;

R² and R⁵ are each hydrogen;

R⁷ is a terminal COOR⁶ group, wherein R⁶ is hydrogen or a C₁-C₂₄ alkyl; and

R³ and R⁴ are each independently, hydrogen, oxygen, C₁-C₂₄ alkyl, NO₂, OH, ONO₂, NO, ONO or OOH, provided at least one of R³ or R⁴ is NO₂ and the other of R³ or R⁴ is hydrogen, ONO or ONO₂.

In certain embodiments of formula III, R¹ is C₁-C₂₄ alkyl, more particularly C₃-C₂₀ alkyl; R⁶ is hydrogen; R³ is NO₂ and R⁴ is ONO₂ or R⁴ is NO₂ and R³ is ONO₂.

Another illustrative compound that can be stabilized as described herein is a compound comprising a dicarboxylic acid of a structure of formula IV:

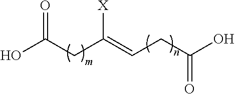

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂,

m is from 1 to 10; and

n is from 1 to 10.

In certain embodiments of formula IV, X is —NO₂.

A further illustrative compound that can be stabilized as described herein is a compound comprising a dicarboxylic acid of a structure of formula V:

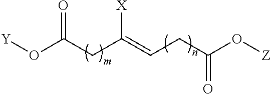

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;

Y and Z are each, independently, hydrogen or a C₁ to C₁₀ alkyl;

m is from 1 to 10; and

n is from 1 to 10.

In certain embodiments of formula V, X is —NO₂, and at least one Y and Z is a C₁ to C₄ alkyl.

An additional illustrative compound that can be stabilized as described herein is a compound comprising a dicarboxylic acid of a structure of formula VI:

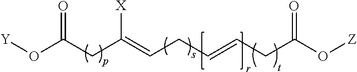

wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;

Y and Z are each, independently, hydrogen or C₁ to C₁₀ alkyl;

p and t are each, independently, 1 to 10;

s is absent or 1 to 10, and

r is 1.

In certain embodiments, the nitroalkene fatty acid is 10-nitro-octadec-9-enoic acid (10-NO₂-OA).

In certain embodiments, the nitroalkene fatty acid is 9-nitro-octadec-9-enoic acid (9-NO₂-OA).

In certain embodiments, the nitroalkene fatty acid is 8-nitro-nonadec-9-enoic acid.

In certain embodiments, the nitroalkene fatty acid is 7-NO₂-nonadec-7-enoic acid.

In certain embodiments, the nitroalkene fatty acid is 5-NO₂-eicos-5-enoic acid or 6-NO₂-eicos-5-enoic acid.

In certain embodiments, the nitroalkene fatty acid is 9-nitrooctadeca-9,11-dienoic acid In certain embodiments, the nitroalkene fatty acid is 12-nitrooctadeca-9,11-dienoic acid

In certain embodiments, the nitroalkene fatty acid is 9-nitro-12-(nitrooxy)octadec-10-enoic acid.

In certain embodiments, the nitroalkene fatty acid is 12-nitro-9-(nitrooxy)octadec-10-enoic acid.

In certain embodiments, the nitroalkene is substantially pure. In this aspect, the stereochemistry about the carbon-carbon double bond is substantially cis (or Z) or substantially trans (or E).

Illustrative cyclodextrins include α-cyclodextrin, β-cyclodextrin, γ-cyclodextrin, (2-hydroxypropyl)-β-cyclodextrin, (2-hydroxypropyl)-γ-cyclodextrin, and methyl-β-cyclodextrin. β-cyclodextrin is a preferred cyclodextrin.

The amount of amount of active compound mixed with cyclodextrin may vary. In certain embodiments, the molar ratio of nitroalkene fatty acid/cyclodextrin may range from 1:2 to 1:12, more particularly 1:2 to 1:8, and most particularly 1:2 to 1:4.

In certain embodiments, the active compound and the cyclodextrin may be contacted together at a temperature of 10 to 90° C., more particularly 20 to 50° C., and most particularly 30 to 50° C., for forming the complex.

In certain embodiments, the active compound and the cyclodextrin may be contacted together for 1 to 48 hours, more particularly 8 to 16 hours, and most particularly 10 to 16 hours, for forming the complex.

In certain embodiments, the complexes (via the active compound in the complex) disclosed herein may be used for treating a condition in a subject in need thereof. The condition to be treated may be, for example, inflammatory conditions, immune diseases, psoriasis, obesity, metabolic syndrome, acute kidney disease, chronic kidney disease, focal segmental glomerulosclerosis, atherogenesis, adipogenesis, neointimal proliferation, kidney I/R and xenobiotic injury, focal myocardial I/R injury, Ang II-induced systemic hypertension, pulmonary hypertension, cancer, cardiac and pulmonary fibrosis, liver fibrosis, non-alcoholic steatohepatitis (NASH), non-alcoholic fatty liver disease (NAFLD), breast cancer, ovarian cancer, inflammatory bowel disease, nociception, stroke, motor neuron degeneration, diabetes, aneurysm, aortic stiffness, lupus erythematosus, STING-associated vasculopathy with onset in infancy (SAVI), asthma, chronic obstructive pulmonary disease (COPD), and focal segmental glomerulosclerosis

In certain embodiments, the inflammatory condition may be organ preservation for transplantation, osteoarthritis, chronic obstructive pulmonary disease (COPD), atherosclerosis, hypertension, allograft rejection, pelvic inflammatory disease, ulcerative colitis, Crohn's disease, allergic inflammation in the lung, cachexia, stroke, congestive heart failure, pulmonary fibrosis, hepatitis, glioblastoma, Guillain-Barre Syndrome, systemic lupus erythematosus viral myocarditis, posttransplantation organ protection, acute pancreatitis, irritable bowel disease general inflammation, autoimmune disease, autoinflammatory disease, arterial stenosis, organ transplant rejection and bums, chronic lung injury and respiratory distress, insulin-dependent diabetes, non-insulin dependent diabetes, hypertension, obesity, arthritis, neurodegenerative disorders, lupus, Lyme's disease, gout, sepsis, hyperthermia, ulcers, enterocolitis, osteoporosis, viral or bacterial infections, cytomegalovirus, periodontal disease, glomerulonephritis, sarcoidosis, lung disease, lung inflammation, fibrosis of the lung, asthma, acquired respiratory distress syndrome, tobacco induced lung disease, granuloma formation, fibrosis of the liver, graft vs. host disease, postsurgical inflammation, coronary and peripheral vessel restenosis following angioplasty, stent placement or bypass graft, coronary artery bypass graft (CABG), acute and chronic leukemia, B lymphocyte leukemia, neoplastic diseases, arteriosclerosis, atherosclerosis, myocardial inflammation, psoriasis, immunodeficiency, disseminated intravascular coagulation, systemic sclerosis, amyotrophic lateral sclerosis, multiple sclerosis, Parkinson's disease, Alzheimer's disease, encephalomyelitis, edema, inflammatory bowel disease, hyper IgE syndrome, cancer metastasis or growth, adoptive immune therapy, reperfusion syndrome, radiation bums, alopecia areta, ischemia, myocardial infarction, artelial stenosis, rheumatoid arthritis, coronary restenosis, neurocognitive decline and insulin resistance.

In embodiments described herein, the method of treating inflammation, obesity, metabolic syndrome, acute kidney disease, and chronic kidney disease comprises administering to a subject in need thereof an effective amount of the complex and, optionally, a pharmaceutically acceptable excipient.

In embodiments described herein, the method of treating inflammation, obesity, metabolic syndrome, focal segmental glomerulosclerosis, non-alcoholic steatohepatitis (NASH), non-alcoholic fatty liver disease (NAFLD), alcoholic fatty liver disease (AFLD), acute kidney disease, lithium-induced nephropathy, and chronic kidney disease comprises administering to a subject in need thereof an effective amount of the complex and, optionally, a pharmaceutically acceptable excipient, wherein the complex provides release of an activated fatty acid.

In certain embodiments, the complexes disclosed herein are useful for treating endotoxin-induced vascular inflammation, endotoxemia and multi-organ injury, inflammatory bowel disease (IBD), allergic airway disease, renal ischemia and reperfusion (I/R) injury, diabetic kidney disease, pulmonary arterial hypertension (PAH), myocardial I/R injury, hypertension, and atherosclerosis.

In some embodiments, the methods disclosed herein involve administering to a subject in need of treatment a pharmaceutical composition, for example a composition that includes a pharmaceutically acceptable carrier and a therapeutically effective amount of one or more of the complexes disclosed herein. The complexes may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The complexes are preferably formulated into suitable pharmaceutical preparations such as tablets, capsules, or elixirs for oral administration or in sterile solutions, emulsions or suspensions for parenteral or topical administration or inhalation.

One embodiment disclosed herein is a pharmaceutical composition that includes a complex of cyclodextrin and an active compound that can be resuspended in water and administered orally.

Also disclosed is a pharmaceutical composition that includes a complex of cyclodextrin and an active compound in a powder form, which can be solvated for administration to infants, toddlers and children (e.g., age 12 and under) as a liquid medicine.

In some embodiments, one or more of the disclosed complexes are mixed or combined with a suitable pharmaceutically acceptable carrier to prepare a pharmaceutical composition. Pharmaceutical carriers or vehicles suitable for administration of the complexes provided herein include any such carriers known to be suitable for the particular mode of administration. Remington: The Science and Practice of Pharmacy, The University of the Sciences in Philadelphia, Editor, Lippincott, Williams, & Wilkins, Philadelphia, Pa., 21^{st} Edition (2005), describes exemplary compositions and formulations suitable for pharmaceutical delivery of the complexes disclosed herein. In addition, the complexes may be formulated as the sole pharmaceutically active ingredient in the composition or may be combined with other active ingredients.

Upon mixing or addition of the complex(es) to a pharmaceutically acceptable carrier, the resulting mixture may be a solution, suspension, emulsion, dry powder pills, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the complex in the selected carrier or vehicle. Where the complexes exhibit insufficient solubility, methods for solubilizing may be used. Such methods are known and include, but are not limited to, using co-solvents such as dimethylsulfoxide (DMSO), using surfactants such as Tween®, and dissolution in aqueous sodium bicarbonate. The disclosed complexes may also be prepared with carriers that protect them against rapid elimination from the body, such as time-release formulations or coatings. Such carriers include controlled release formulations, such as, but not limited to, microencapsulated delivery systems. The disclosed complexes and/or compositions can be enclosed in multiple or single-dose containers. The complexes and/or compositions can also be provided in kits, for example, including component parts that can be assembled for use. For example, one or more of the disclosed complexes may be provided in a lyophilized form and a suitable diluent may be provided as separated components for combination prior to use. In some examples, a kit may include a disclosed complex and a second therapeutic agent for co-administration. The complex and second therapeutic agent may be provided as separate component parts. A kit may include a plurality of containers, each container holding one or more unit dose of the complex. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The pharmaceutical compositions may be in a dosage unit form such as an injectable fluid, an oral delivery fluid (e.g., a solution or suspension), a nasal delivery fluid (e.g., for delivery as an aerosol or vapor), a semisolid form (e.g., a topical cream), or a solid form such as powder, pill, tablet, or capsule forms.

The complex is included in the pharmaceutically acceptable carrier in an amount sufficient to exert a therapeutically useful effect in the absence of undesirable side effects on the subject treated. A therapeutically effective concentration may be determined empirically by testing the complex in known in vitro and in vivo model systems for the treated disorder. In some examples, a therapeutically effective amount of the complex is an amount that lessens or ameliorates at least one symptom of the disorder for which the complex is administered. Typically, the compositions are formulated for single dosage administration. The concentration of complex in the drug composition will depend on absorption, inactivation, and excretion rates of the active compound, the dosage schedule, and amount administered as well as other factors known to those of skill in the art.

In some examples, about 1 mg to 5000 mg of a disclosed complex, a mixture of such complexes, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 5000 mg (for example, about 5 mg to about 1000 mg, about 10 mg to 500 mg, about 30 mg to 300 mg, or about 50 mg to 100 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 500 mg, about 700 mg, about 800 mg, about 1000 mg, about 2000 mg about 3000 mg, about 5000 mg, or more of the disclosed complex(es).

The disclosed complexes or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate-release tablets, these compositions may contain microcrystalline cellulose, dicalcium phosphate, starch, magnesium stearate and lactose and/or other excipients, binders, extenders, disintegrants, diluents and lubricants. If oral administration is desired, the complex is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the complex can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

Dosage unit forms can contain various other materials, which modify the physical form of the dosage unit, for example, coatings of sugar and other enteric agents. The complexes can also be administered as a component of an elixir, suspension, syrup, wafer, chewing gum or the like. A syrup may contain, in addition to the active ingredient, sucrose as a sweetening agent and certain preservatives, dyes and colorings, and flavors.

When administered orally, the complex can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the complex can be administered orally to humans in a dosage range of 0.1 to 100 mg/kg body weight in single or divided doses. One illustrative dosage range is 1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific complex employed, the metabolic stability and length of action of that complex, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The complex can be administered parenterally, for example, by IV, IM, depo-IM, SC, or depo-SC. When administered parenterally, a therapeutically effective amount of about 1 to about 5000 mg/day (such as about 5 mg/day to about 1000 mg/day, or about 20 mg/day to about 200 mg/day) may be delivered. When a depot formulation is used for injection once a month or once every two weeks, the dose may be about 1 mg/day to about 5000 mg/day, or a monthly dose of from about 30 mg to about 15000 mg.

The complex can also be administered sublingually. When given sublingually, the complex should be given one to four times daily in the amounts described above for IM administration.

The complex can also be administered intranasally. When given by this route, the appropriate dosage forms are a nasal spray or dry powder. The dosage of the complex for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The complex can be administered intrathecally. When given by this route, the appropriate dosage form can be a parenteral dosage form. The dosage of the complex for intrathecal administration is the amount described above for IM administration.

The complex can be administered topically. When given by this route, the appropriate dosage form is a cream, ointment, or patch. When administered topically, an illustrative dosage is from about 2 mg/day to about 1000 mg/day. Because the amount that can be delivered by a patch is limited, two or more patches may be used.

The complex can be administered rectally by suppository. When administered by suppository, an illustrative therapeutically effective amount may range from about 2 mg to about 2000 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular complex administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### EXAMPLES

As a first step to approach inclusion complex formation, a conjugated linoleic acid was used as a surrogate for the highly unstable NO₂-OA to ascertain whether there was a preferred form of cyclodextrin for higher yields as well as evaluate the effect of molar ratios on the formation of the inclusion complexes. NO₂-OA requires storage at −80° C., and is labile to temperatures gradually increasing decomposition rates at temperatures above −20° C., presence of water and humidity levels (e.g., atmospheric moisture), the presence of nucleophiles, and the presence protein amino acids. This includes decomposition induced by the shell from hard gelatin capsules. The instability results from the reaction with nucleophiles which promotes the decomposition reactions including the isomerization of the nitroalkene C-C double bond, double bond migration, dimerization reaction between two NO₂-OA molecules, oxidation.
1. The CLA-Cyclodextrin complex was formed by initially weighting 499.39 mg of β-Cyclodextrin (0.44 mmol) and dissolving it in 3 ml of water by mixing and heating to 50 C. 61.69 mg of CLA (0.22 mmol) were weighted in a clean tube and dissolved in ethanol to be slowly then added to the solution containing the β-cyclodextrin. The mixture was left to overnight at 37 C under mild agitation to form the inclusion complexes. The mixture was then dried under a stream of nitrogen for 20-25 min and the resulting solution was placed in a −80 C freezer to freeze. The frozen solution was placed in a liophylizer and lipophylized overnight. The resulting powder was transferred to a clean tube and kept at 4 C. CLA content was quantified by HPLC-UV and shown to contain ˜107 ug CLA/mg inclusion complex.
2. Second, and as NO₂-OA is our target for inclusion complex stabilization is a nitrated oleic acid), oleic acid (OA) was used to test the formation of inclusion complexes. Again, β-cyclodextrin complexes were superior and the following protocol was used.
3. The OA-β-Cyclodextrin complex was formed by initially weighing 499.39 mg of β-Cyclodextrin (0.44 mmol) and dissolving it in 3 ml of water by mixing and heating to 50 C. 62.14 mg of CLA (0.22 mmol) were weighted in a clean tube and dissolved in ethanol to then be slowly added to the solution containing the β-cyclodextrin. The mixture was left to overnight at 37 C under mild agitation to form the inclusion complexes. The mixture was then dried under a stream of nitrogen for 20-25 min and the resulting solution was placed in a −80 C freezer to freeze. Frozen solution was placed in a lyophilizer and lypophylized overnight. The resulting powder was transferred to a clean tube and kept at 4 C. OA content was quantified by HPLC-UV and shown to contain ˜99 ug OA/mg inclusion complex.

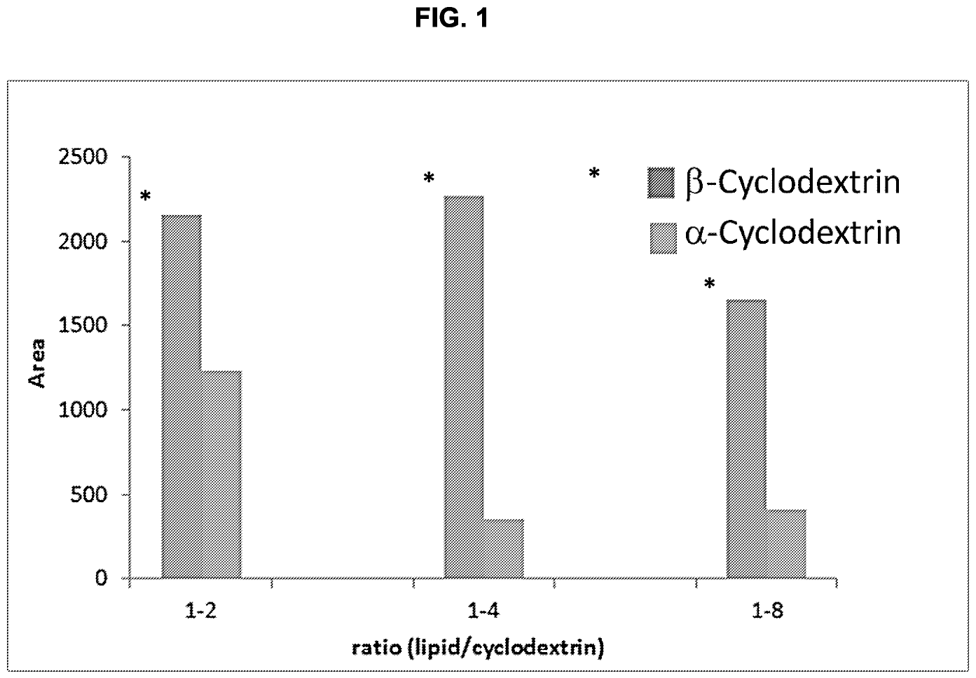

It was discovered that β-cyclodextrin more effectively accommodated the conjugated linoleic acid than α-cyclodextrin. Lower yields of incorporation into inclusion complexes were obtained with α-cyclodextrin. The results are shown in FIG. 1.

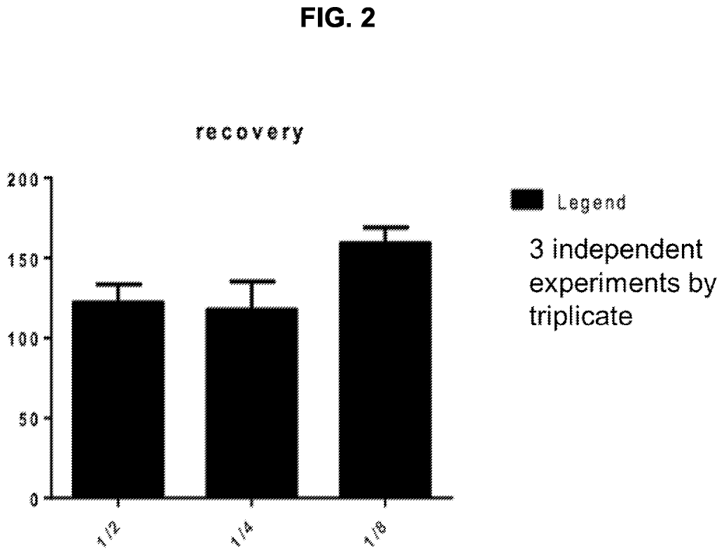

Once it was determined that β-cyclodextrin was superior to α-cyclodextrin, different molar ratios of NO₂-OA to β-cyclodextrin were tested. Three independent analyses were performed using three different molar ratios between NO₂-OA and β-cyclodextrin. The tested molar ratios between NO₂-OA and β-cyclodextrin were 1:2, 1:4 and 1:8. The data show that increasing the molar ratios of β-cyclodextrin relative to NO₂-OA has only a minor effect on total recovery (see FIG. 2).

The different ratio complexes were obtained by dissolving the different required amount of β-cyclodextrin in the initial 3 ml of water. Thus, for a 1:4, and 1:8 molar ratios, 998.8 and 1997.6 mg respectively. In some cases, in particular when higher molar ratios were used, increased heat was necessary to initially dissolve the β-cyclodextrin in water. In the case of 1:8 molar ratio, up to 75 C were used and with up to 30 min incubation/mixing time.

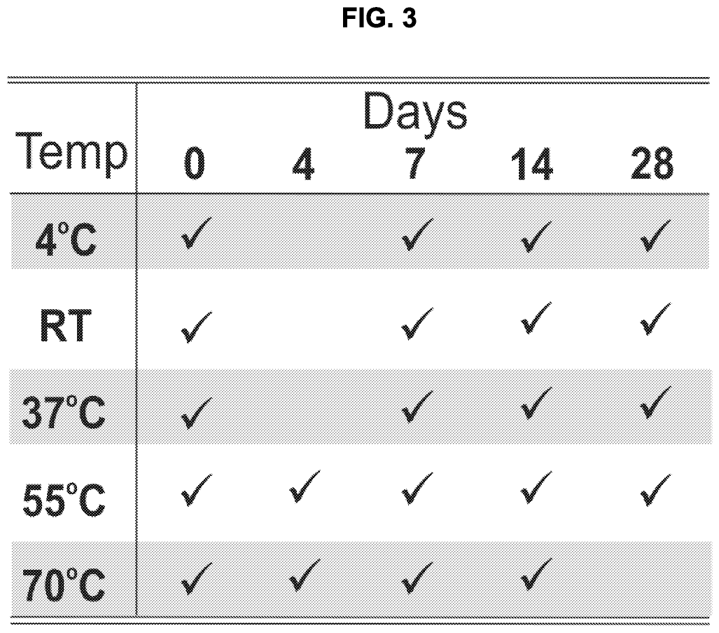

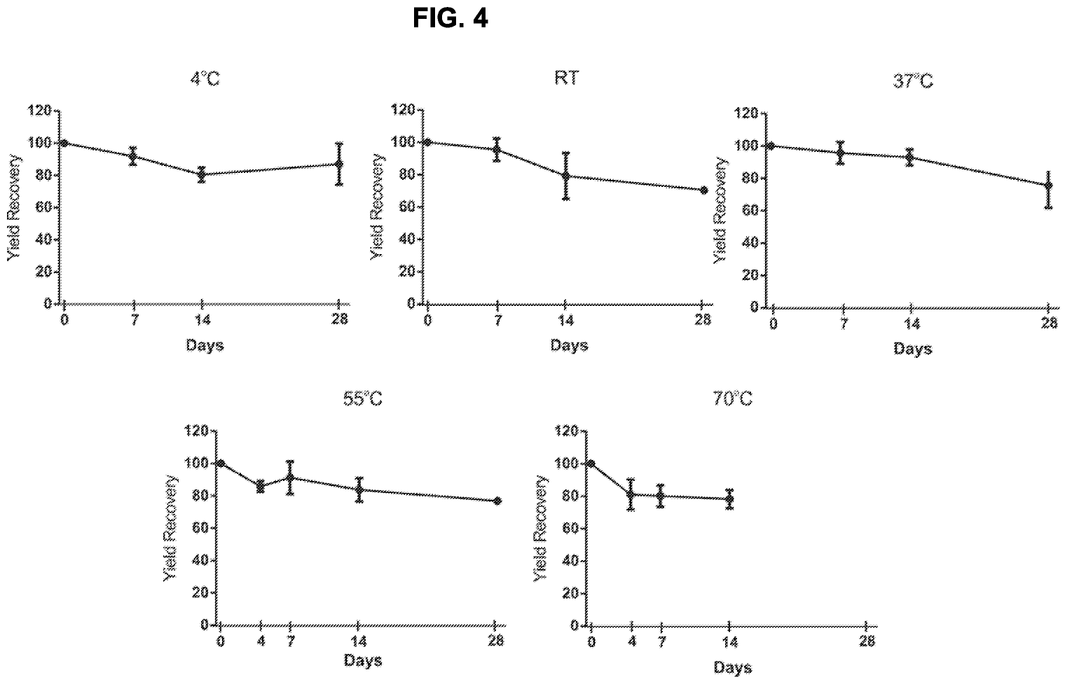

Conventionally, NO₂-OA has to be maintained at −80° C. To test whether the β-cyclodextrin-NO₂-OA inclusion complex disclosed herein would provide stability as a dry powder (after lyophilization), it was subjected to the stability evaluation shown in FIG. 3 and the results are shown in FIG. 4.

The NO₂-OA/η-cyclodextrin inclusion complex at a 1:2 molar ratio had significant thermal stability. NO₂-OA, if not stabilized with β-cyclodextrin would result in a significant loss over 4 weeks at 70° C. Conventionally, NO₂-OA is stabilized using oils as the only way to marginally improve stability. The conventional approaches used to stabilize NO₂-OA are all based on solvation in oily viscous liquid formulations that are not amenable to use as a formulation for humans given the low stability and consequently low shelf life. Oils that are used included olive oil, sesame oil and synthetic oils (synthetic triacylglycerols).

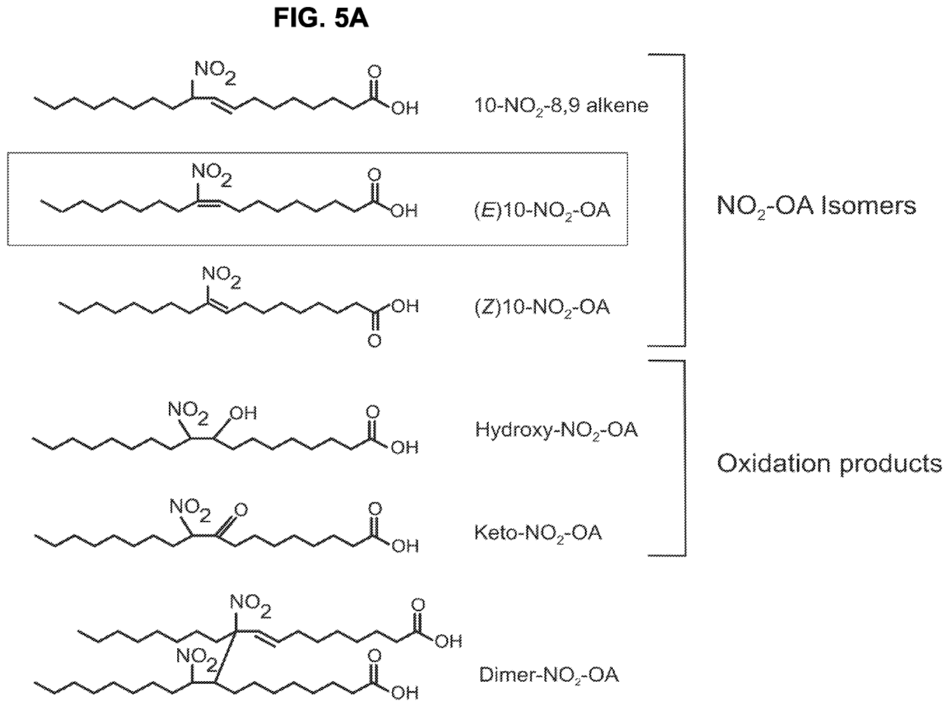

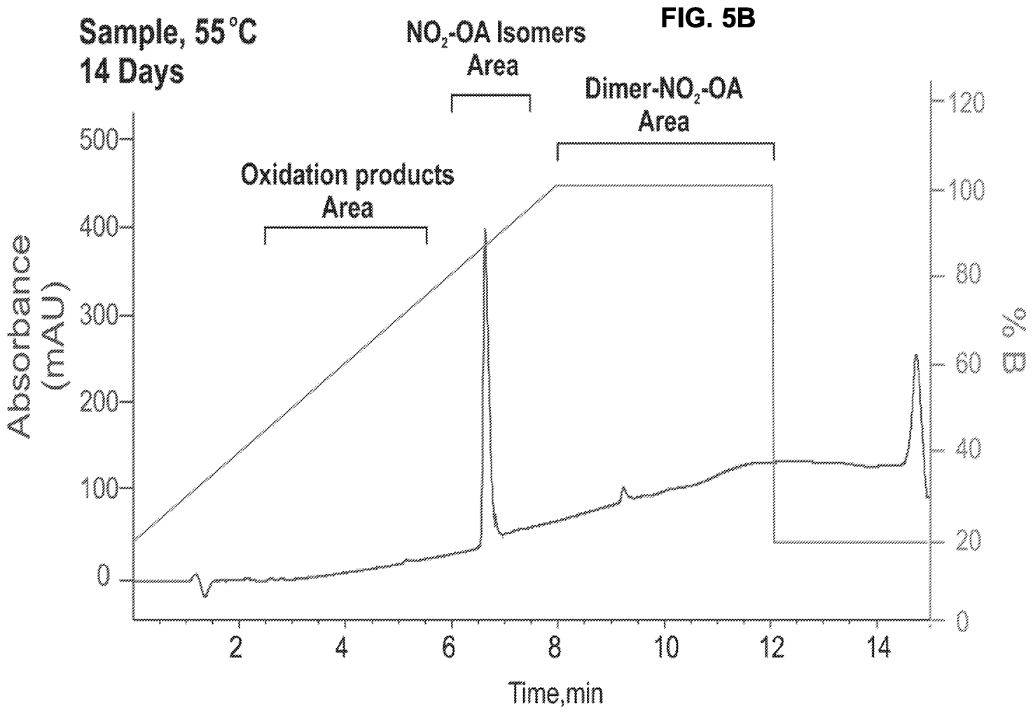

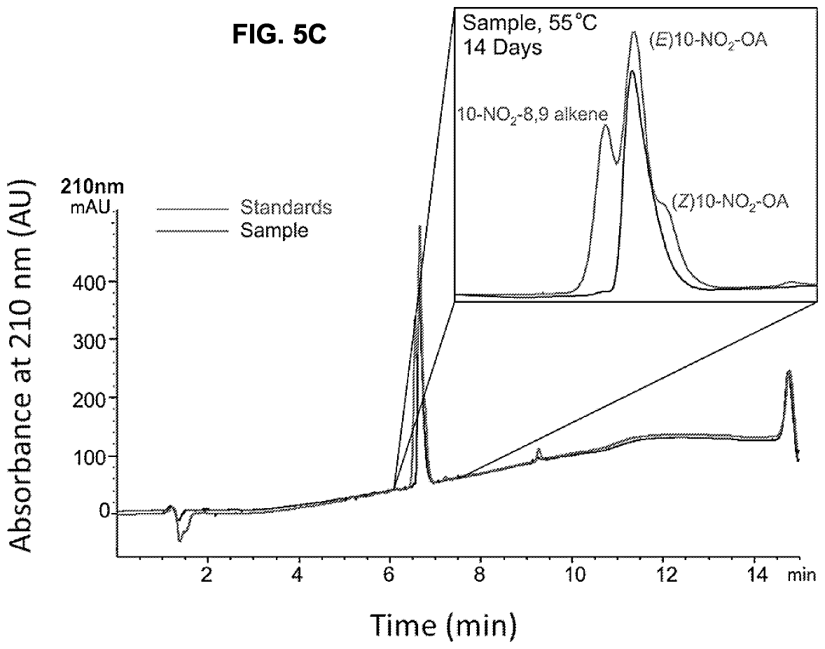

The decomposition of NO₂-OA at high temperatures is a process that leads to several decomposition products that include E isomerization to the Z isomer (trans), the formation of OH-NO₂-OA, oxo-NO₂-OA, and the dimer of NO₂-OA. None of these decomposition products depicted in FIG. 5A were identified in the chromatogram evaluated 14 days after exposure of NO₂-OA/β-cyclodextrin to 55° C. (see FIG. 5B). The small peak evident at ˜9 min was also present in the initial NO₂-OA stock solution, did not change intensity after incubation and does not correspond to the formation of dimer during incubation.

To support the fact that β-cyclodextrin inclusion complexes are protective against temperature-dependent E to Z isomerization, or further double bond migration, a chromatographic analysis was performed. FIG. 5A shows the structures of possible decomposition products and FIG. 5B shows the chromatogram. When a mixture containing (E)10-NO₂-OA, (Z)10-NO₂-OA, and 10-NO₂-octadec-8-enoic acid was resolved on a C₁₈ Polaris column and followed at 210 nm, a main peak for (E)10-NO₂-OA was observed, with a shoulder before the main peak corresponding to the 10-NO₂-octadec-8-enoic acid and a shoulder after corresponded to the more linear (Z)10-NO₂-OA (see FIG. 5C). As shown in FIG. 5B-C, the blue trace corresponding to the 10-NO₂-OA obtained from the inclusion complex after exposure to 55° C. for 14 days showed no evidence of these decomposition products.

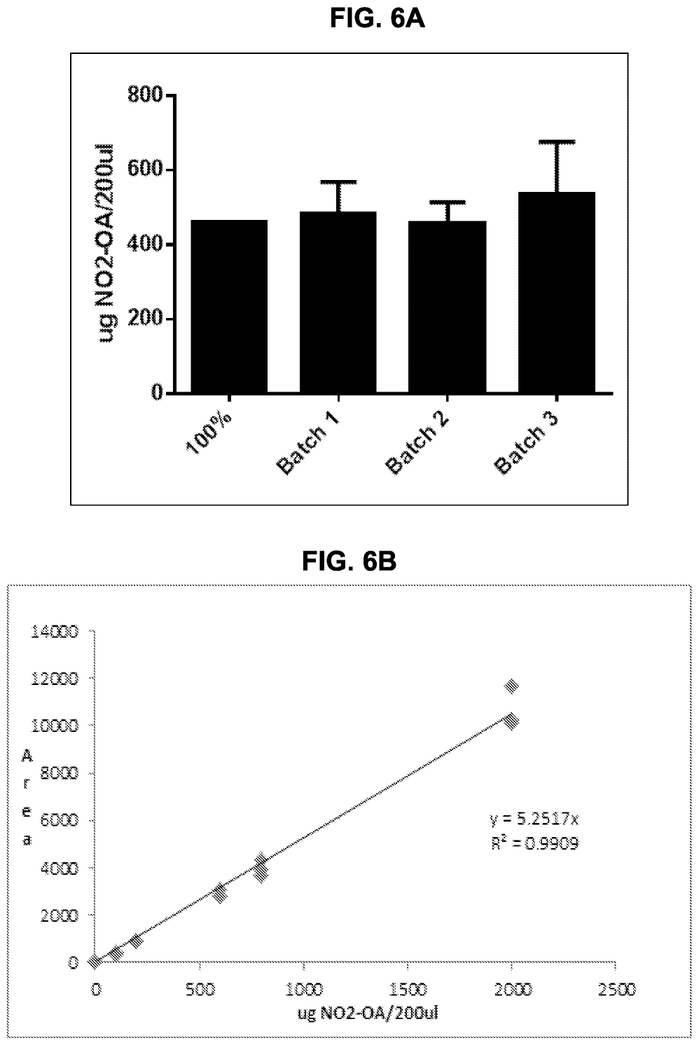

The method used to generate the inclusion complex is high in yield and very reproducible. Three independent batches provided an overall yield of 100% when compared to the initial material used in the preparation (left column) (see FIG. 6A). Quantification of the yield of incorporation was performed by running external standard curves on the HPLC-UV at 210 nm using 10-NO₂-OA standards in ethanol as shown in FIG. 6B.

The decrease in total quantity observed after 14-28 days of exposure at higher temperatures was also observed at lower temperatures and at 4° C. (FIG. 4). This is not related to the decomposition of the material as shown by the absence of the main oxidation/degradation products, but might be a consequence of changes in the physical properties of the complex. During the method development, it was observed that the quantification of these complexes is highly influenced by the extraction method used to quantify remaining NO₂-OA. In this regards, different extraction methods that included different organic solvent yielded different overall results, even when starting from the same inclusion complex stock material.

An important consideration when working with nitroalkene fatty acids is their high sensitivity to water or humidity. To demonstrate that the NO₂-OA/β-cyclodextrin complex could be used to develop a stable solution of NO₂-OA, the NO₂-OA/β-cyclodextrin inclusion complex was resuspended in water and its stability was measured. Stability was compared with the total amount added to the aqueous solution.

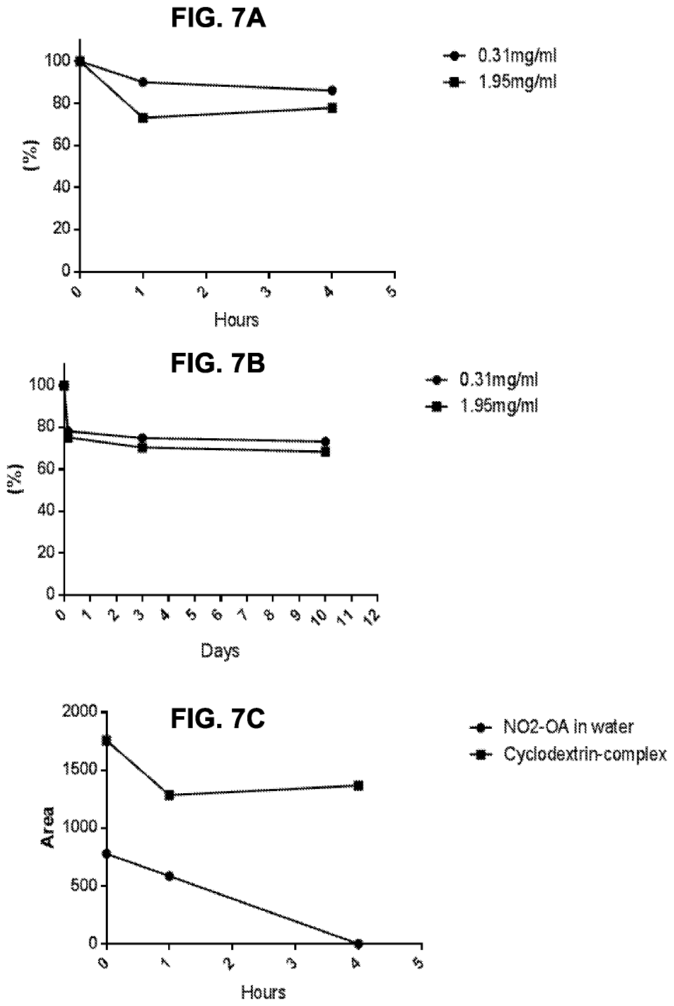

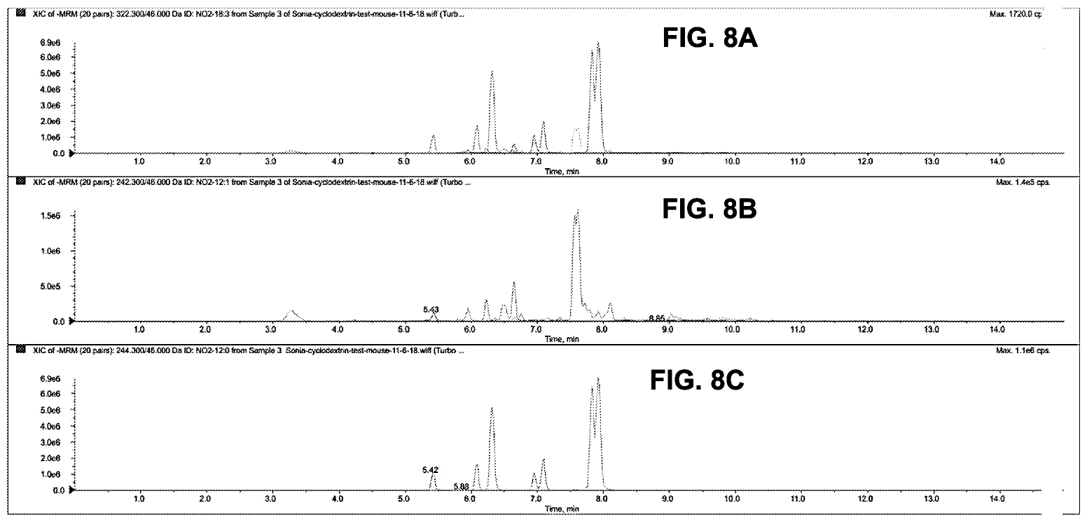

FIGS. 7A and 7B show that after a small decay caused by the dissolution that occurs in the first hour, the NO₂-OA is perfectly stable in water for periods of time longer than 10 days. These stability assays were performed at room temperature and data was similar between the two tested concentrations of 0.31 mg/ml and 1.95 mg/ml. Since no decomposition products are observed in the water solution, it is proposed that the initial decrease is related to loosely bound inclusion complexes that decompose under aqueous conditions. A new equilibrium is rapidly achieved and the sample is stable after this initial decay.

As a comparison, the addition of NO₂-OA to water results in complete decomposition of NO₂-OA in water within 4 hours after of addition of NO₂-OA (see FIG. 7C). Thus, inclusion complexes can be utilized to both stabilize the NO₂-OA for storage/formulation (as a stable powder) but also as a vehicle to effectively formulate the NO₂-OA as a stable liquid drug upon addition of water.

The NO₂-OA/β-cyclodextrin complex was administered to mice as a suspension in water. To check that this is a viable vehicle, taste aversion was tested by measuring water consumption. Nitrated fatty acids are activators of TRP channels that usually detect the presence of pungent compounds in spicy foods containing capsaicin or related electrophiles. As such, nitrated fatty acids are compounds that might elicit a strong taste aversion because of the spicy and pungent sensation they may induce. The β-cyclodextrin inclusion complex was effective in masking this response and leads to no changes in water consumption by mice.

To check that the β-cyclodextrin inclusion complexes are absorbed when consumed orally from drinking liquids having a suspended NO₂-OA/β-cyclodextrin complex , an analysis of the plasma and feces after drinking a suspension that would provide the mice with a daily dose of 10 and 50 mg/kg resulted in the detection of the main metabolites. This shows that β-cyclodextrin complexes are not only an effective way to stabilize the metabolites for capsule or pill formulation but also to generate liquid pediatric formulations amenable for administration to children (e.g., age 12 and under). The β-cyclodextrin inclusion complexes are effectively degraded during digestion and released to be absorbed, metabolized and excreted.

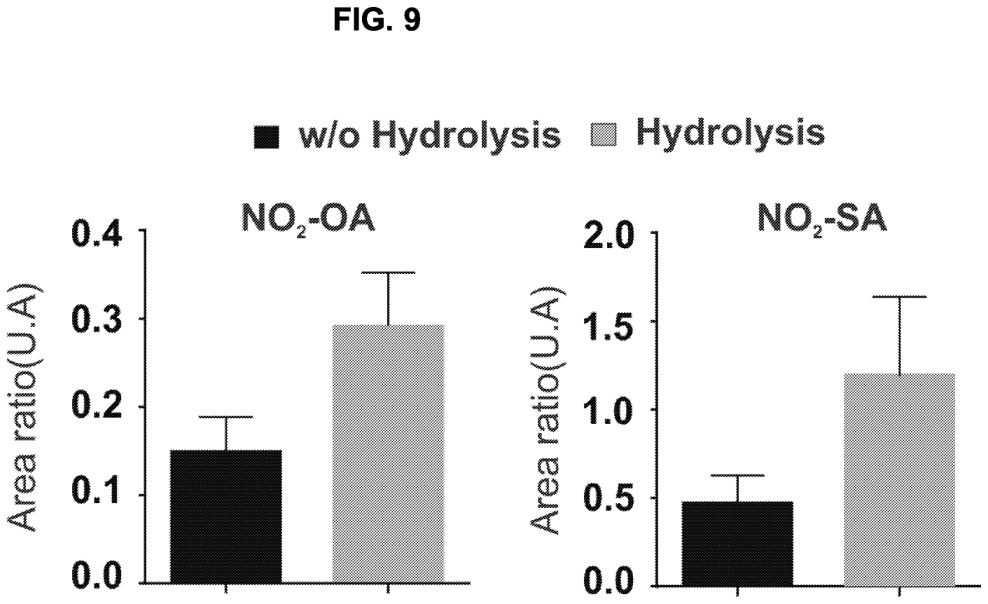

NO₂-OA was stabilized as an inclusion complex with β-cyclodextrin as disclosed herein and delivered to mice in drinking water. The mice readily drank the water without any change in their drinking habits or daily fluid intake, indicating taste masking by the β-cyclodextrin inclusion complex. Levels of nitro oleic acid were measured in plasma (NO₂-OA) before and after hydrolysis of complex lipids (mostly triglycerides). See FIG. 9. This indicates that as previously observed with oral NO₂-OA dissolved in oil, absorbed nitrated fatty acid follow a similar incorporation into complex lipids and biodistribution. Higher levels of NO₂-SA were detected as previously observed (right figure).

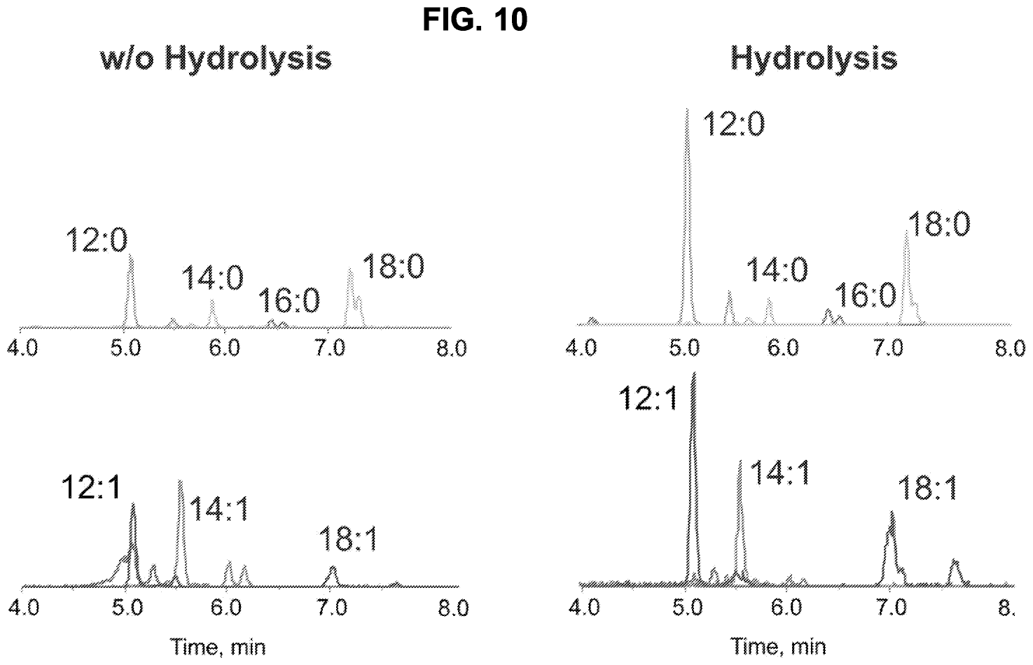

FIG. 10 shows an analysis of nitro oleic acid profile as well as its main reported metabolites. NO₂-OA was stabilized as an inclusion complex with β-cyclodextrin as disclosed herein and delivered to mice in drinking water to obtain daily doses of 10 and 50 mg/kg. The mice readily drank the water at both concentrations without any change in their drinking habits or daily fluid intake. Plasma metabolite profiles show absorption and metabolism of NO₂-OA. Upper panels show metabolites obtained following reduction of the nitroalkene double bond (18:0) and after β-oxidation of the terminal carboxylic acid (NO₂-12:0, NO₂-14:0. NO₂-16:0). Lower panels show the metabolites corresponding to two and three β-oxidation cycles of the carboxylic acid end of NO₂-OA (14:1 and 12:1). Left panels show NO₂-OA and its metabolites as free acids in plasma. Right panel shows total metabolites after complete hydrolysis of plasma lipids.

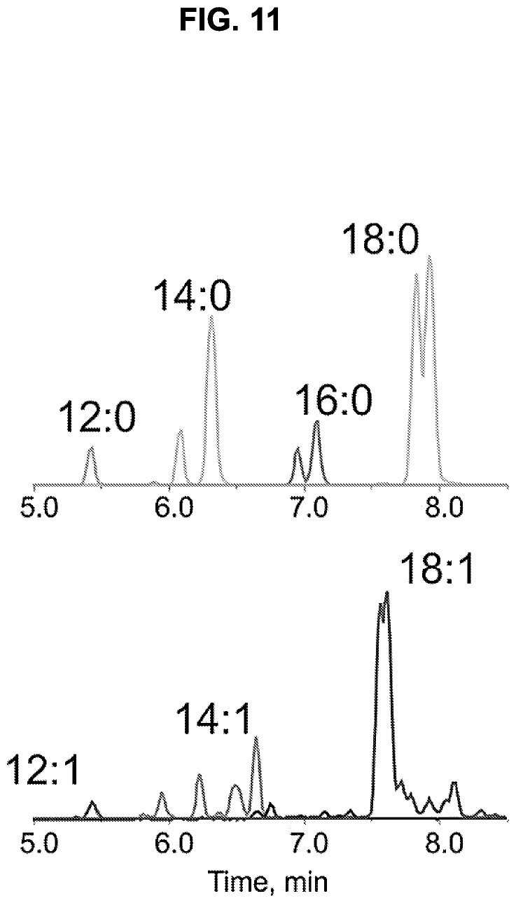

FIG. 11 shows an analysis of NO₂-OA profile of mice feces as well as its main reported metabolites. NO₂-OA was stabilized as an inclusion complex with β-cyclodextrin as disclosed herein and delivered to mice in drinking water to obtain daily doses of 10 and 50 mg/kg. In this case a dose of 50 mg/kg is shown. Feces metabolite profile shows uptake of nitro oleic acid and extensive metabolism. It has been reported that a large amount of NO₂-OA is excreted through the feces as NO₂-OA and as partially metabolized material. This further that stabilized inclusion complexes can be solvated and administered to reach central circulation and display a predicted metabolic profile both in urine and in feces. Upper panels shows β-oxidation of reduced metabolites while the lower panel shows β-oxidation of the parent compound.

An example of making a NO₂-OA/β-cyclodextrin complex is described below:
1. Weigh 71.94 mg of nitrated oleic acid (NO₂-OA) (0.22 mmol) in a clean tube
2. Weigh 499.39 mg of β-Cyclodextrin (0.44 mmol)
3. In a tube put the β-Cyclodextrin and add 3 ml of water, mix very well using a vortex for 1 minute to obtain a suspension. Heat to 60° C. for 15 minutes. This will dissolve the β-cyclodextrin.
4. Take the weighted NO₂-OA and add lml of ethanol and mix well using a vortex for 1 minute.
5. Add the NO₂-OA solution (NO₂-OA in ethanol) to the tube containing the β-Cyclodextrin and the water.
6. Allow the inclusion complex to form during 16 h at 37° C. using rotatory agitation.
7. Take the tube with the inclusion complex (NO₂-OA/β-Cyclodextrin).
8. Dry under nitrogen stream until ethanol is evaporated. This step is performed at room temperature and takes around 20-25 min.
9. Take the inclusion complex and freeze to −80° C. and leave there for an hour.
10. Take the tube containing the frozen inclusion complex and lyophilize overnight.
11. Take the powder and keep at 4° C.

In view of the many possible embodiments to which the principles of the disclosed invention may be applied, it should be recognized that the illustrated embodiments are only preferred examples of the invention and should not be taken as limiting the scope of the invention.

1. A composition comprising a complex of a cyclodextrin with a nitroalkene.

2. A composition comprising a complex of a cyclodextrin with an active compound, wherein the active compound is:
a nitroalkene is a structure of formula I: 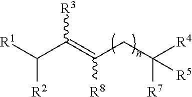
wherein R¹ is hydrogen, C₁-C₂₄ alkyl, C₁-C₂₄ alkenyl, or C₁-C₂₄ alkynyl;
R², R³, R⁷, and R⁸ are each independently, hydrogen, oxygen, C₁-C₂₄ alkyl, NO₂, OH, or OOH;
R⁴ is a terminal COOR⁶ group, wherein R⁶ is hydrogen, or a C₁-C₂₄ alkyl;
R⁵ is hydrogen, C₁-C₂₄ alkyl, or R⁴ and R⁵ collectively form ═C(R⁹)(R¹⁰), wherein R⁹ comprises C₁-C₂₄ alkyl, C₁-C₂₄ alkenyl, or C₁-C₂₄ alkynyl, or wherein R⁹ is a terminal COOR⁶ group, and R¹⁰ is hydrogen, NO₂, OH, or OOH;
n is from 1 to 24; and
wherein the nitroalkene fatty acid includes at least one NO₂ group;
a nitroalkene is a structure of formula II: 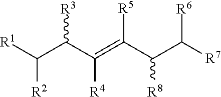
wherein R¹ is hydrogen, C₁-C₂₄ alkyl, C₁-C₂₄ alkenyl, or C₁-C₂₄ alkynyl;
R², R⁴, R⁵ and R⁶ are each hydrogen;
R⁷ is a terminal COOR^{S} group, wherein R⁹ is hydrogen or a C₁-C₂₄ alkyl; and
R³ and R⁸ are each independently, hydrogen, oxygen, C₁-C₂₄ alkyl, NO₂, OH, ONO₂, NO, ONO or OOH, provided at least one of R³ or R⁸ is NO₂ and the other of R³ or R⁸ is hydrogen, ONO or ONO₂;
a nitro group-containing compound is a structure of formula III: 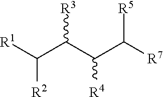
wherein R¹ is hydrogen, C₁-C₂₄ alkyl, C₁-C₂₄ alkenyl, or C₁-C₂₄ alkynyl;
R² and R⁵ are each hydrogen;
R⁷ is a terminal COOR⁶ group, wherein R⁶ is hydrogen or a C₁-C₂₄ alkyl; and
R³ and R⁴ are each independently, hydrogen, oxygen, C₁-C₂₄ alkyl, NO₂, OH, ONO₂, NO, ONO or OOH, provided at least one of R³ or R⁴ is NO₂ and the other of R³ or R⁴ is hydrogen, ONO or ONO₂;
a compound comprising a dicarboxylic acid of a structure of formula IV: 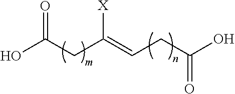
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂,
m is from 1 to 10; and
n is from 1 to 10;
a compound comprising a dicarboxylic acid of a structure of formula V: 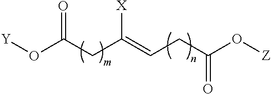
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or a C₁ to C₁₀ alkyl;
m is from 1 to 10; and
n is from 1 to 10; or
a compound comprising a dicarboxylic acid of a structure of formula VI: 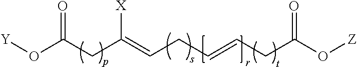
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or C₁ to C₁₀ alkyl;
p and t are each, independently, 1 to 10;
s is absent or 1 to 10, and
r is 1.

3. The composition of claim 1, wherein the composition is in the form of a solid powder.

4. The composition of claim 1, wherein the composition is in a pharmaceutical oral administration dosage form.

5. The composition of claim 2, wherein the composition is in a solid oral administration dosage unit.

6. The composition of claim 1, wherein the cyclodextrin is β-cyclodextrin.

7. The composition of claim 1, wherein the cyclodextrin is α-cyclodextrin, γ-cyclodextrin, (2-hydroxypropyl)-β-cyclodextrin, (2-hydroxypropyl)-γ-cyclodextrin, or methyl-β-cyclodextrin.

8. The composition of claim 2, wherein the active compound is a nitroalkene of formula I.

9. The composition of claim 8, wherein R¹ is C₁-C₂₄ alkyl, R² is hydrogen, one of R³ or R⁸ is NO₂ and the other of R³ or R⁸ is hydrogen, n is 3 to 20, R⁴ is —COOH, R⁵ is hydrogen, and R⁷ is hydrogen.

10. The composition of claim 8, wherein the nitroalkene of formula I is 10-nitro-octadec-9-enoic acid.

11. (canceled)

12. The composition of claim 1, wherein the active compound is selected from 10-nitro-octadec-9-enoic acid; 9-nitro-octadec-9-enoic acid; 8-nitro-nonadec-9-enoic acid; 7-NO₂-nonadec-7-enoic acid; 5-NO₂-eicos-5-enoic acid; 6-NO₂-eicos-5-enoic acid; 9-nitrooctadeca-9,11-dienoic acid; 12-nitrooctadeca-9,11-dienoic acid; 9-nitro-12-(nitrooxy)octadec-10-enoic acid; or 12-nitro-9-(nitrooxy)octadec-10-enoic acid.

13. A liquid composition comprising (a) water and (b) suspended or dissolved in the water, a solid powder comprising a complex of a cyclodextrin with a nitroalkene.

14. A liquid composition comprising (a) water and (b) suspended or dissolved in the water, a solid powder comprising a complex of a cyclodextrin with an active compound, wherein the active compound is:
a nitroalkene is a structure of formula I: 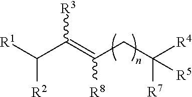
wherein R¹ is hydrogen, C₁-C₂₄ alkyl, C₁-C₂₄ alkenyl, or C₁-C₂₄ alkynyl;
R², R³, R⁷, and R⁸ are each independently, hydrogen, oxygen, C₁-C₂₄ alkyl, NO₂, OH, or OOH;
R⁴ is a terminal COOR⁶ group, wherein R⁶ is hydrogen, or a C₁-C₂₄ alkyl;
R⁵ is hydrogen, C₁-C₂₄ alkyl, or R⁴ and R⁵ collectively form ═C(R⁹)(R¹⁰), wherein R⁹ comprises C₁-C₂₄ alkyl, C₁-C₂₄ alkenyl, or C₁-C₂₄ alkynyl, or wherein R⁹ is a terminal COOR⁶ group, and R¹⁰ is hydrogen, NO₂, OH, or OOH;
n is from 1 to 24; and
wherein the nitroalkene fatty acid includes at least one NO₂ group;
a nitroalkene is a structure of formula II: 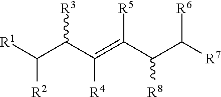
wherein R¹ is hydrogen, C₁-C₂₄ alkyl, C₁-C₂₄ alkenyl, or C₁-C₂₄ alkynyl;
R², R⁴, R⁵ and R⁶ are each hydrogen;
R⁷ is a terminal COOR⁹ group, wherein R⁹ is hydrogen or a C₁-C₂₄ alkyl; and
R³ and R⁸ are each independently, hydrogen, oxygen, C₁-C₂₄ alkyl, NO₂, OH, ONO₂, NO, ONO or OOH, provided at least one of R³ or R⁸ is NO₂ and the other of R³ or R⁸ is hydrogen, ONO or ONO₂;
a nitro group-containing compound is a structure of formula III: 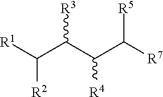
wherein R¹ is hydrogen, C₁-C₂₄ alkyl, C₁-C₂₄ alkenyl, or C₁-C₂₄ alkynyl;
R² and R⁵ are each hydrogen;
R⁷ is a terminal COOR⁶ group, wherein R⁶ is hydrogen or a C₁-C₂₄ alkyl; and
R³ and R⁴ are each independently, hydrogen, oxygen, C₁-C₂₄ alkyl, NO₂, OH, ONO₂, NO, ONO or OOH, provided at least one of R³ or R⁴ is NO₂ and the other of R³ or R⁴ is hydrogen, ONO or ONO₂;
a compound comprising a dicarboxylic acid of a structure of formula IV: 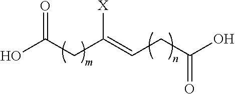
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂,
m is from 1 to 10; and
n is from 1 to 10;
a compound comprising a dicarboxylic acid of a structure of formula V: 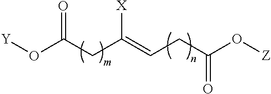
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or a C₁ to C₁₀ alkyl;
m is from 1 to 10; and
n is from 1 to 10; or
a compound comprising a dicarboxylic acid of a structure of formula VI: 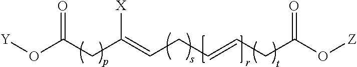
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or C₁ to C₁₀ alkyl;
p and t are each, independently, 1 to 10;
s is absent or 1 to 10, and
r is 1.

15. The liquid composition of claim 13, wherein the cyclodextrin is β-cyclodextrin.

16. The liquid composition of claim 15, wherein the active compound is a nitroalkene of formula I.

17. The liquid composition of claim 16, wherein R¹ is C₁-C₂₄ alkyl, R² is hydrogen, one of R³ or R⁸ is NO₂ and the other of R³ or R⁸ is hydrogen, n is 3 to 20, R⁴ is —COOH, R⁵ is hydrogen, and R⁷ is hydrogen.

18. The liquid composition of claim 16, wherein the nitroalkene of formula I is 10-nitro-octadec-9-enoic acid.

19. (canceled)

20. A complex of a nitroalkene fatty acid and a cyclodextrin.

21. The complex of claim 20, wherein the cyclodextrin is β-cyclodextrin.

22. (canceled)

23. The complex of claim 20, wherein the nitroalkene fatty acid is 10-nitro-octadec-9-enoic acid.

24. A method comprising contacting a nitroalkene with cyclodextrin under conditions resulting in forming a complex of the nitroalkene with the cyclodextrin.

25. A method comprising contacting a cyclodextrin with an active compound under conditions resulting in forming a complex of the cyclodextrin with the active compound, wherein the active compound is:
a nitroalkene is a structure of formula I: 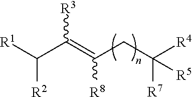
wherein R¹ is hydrogen, C₁-C₂₄ alkyl, C₁-C₂₄ alkenyl, or C₁-C₂₄ alkynyl;
R², R³, R⁷, and R⁸ are each independently, hydrogen, oxygen, C₁-C₂₄ alkyl, NO₂, OH, or OOH;
R⁴ is a terminal COOR⁶ group, wherein R⁶ is hydrogen, or a C₁-C₂₄ alkyl;
R⁵ is hydrogen, C₁-C₂₄ alkyl, or R⁴ and R⁵ collectively form ═C(R⁹)(R¹⁰), wherein R⁹ comprises C₁-C₂₄ alkyl, C₁-C₂₄ alkenyl, or C₁-C₂₄ alkynyl, or wherein R⁹ is a terminal COOR⁶ group, and R¹⁰ is hydrogen, NO₂, OH, or OOH;
n is from 1 to 24; and
wherein the nitroalkene fatty acid includes at least one NO₂ group;
a nitroalkene is a structure of formula II: 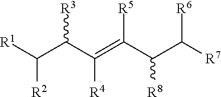
wherein R¹ is hydrogen, C₁-C₂₄ alkyl, C₁-C₂₄ alkenyl, or C₁-C₂₄ alkynyl;
R², R⁴, R⁵ and R⁶ are each hydrogen;
R⁷ is a terminal COOR^{S} group, wherein R⁹ is hydrogen or a C₁-C₂₄ alkyl; and
R³ and R⁸ are each independently, hydrogen, oxygen, C₁-C₂₄ alkyl, NO₂, OH, ONO₂, NO, ONO or OOH, provided at least one of R³ or R⁸ is NO₂ and the other of R³ or R⁸ is hydrogen, ONO or ONO₂;
a nitro group-containing compound is a structure of formula III: 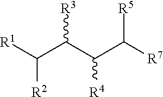
wherein R¹ is hydrogen, C₁-C₂₄ alkyl, C₁-C₂₄ alkenyl, or C₁-C₂₄ alkynyl;
R² and R⁵ are each hydrogen;
R⁷ is a terminal COOR⁶ group, wherein R⁶ is hydrogen or a C₁-C₂₄ alkyl; and
R³ and R⁴ are each independently, hydrogen, oxygen, C₁-C₂₄ alkyl, NO₂, OH, ONO₂, NO, ONO or OOH, provided at least one of R³ or R⁴ is NO₂ and the other of R³ or R⁴ is hydrogen, ONO or ONO₂;
a compound comprising a dicarboxylic acid of a structure of formula IV: 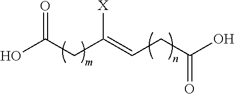
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂,
m is from 1 to 10; and
n is from 1 to 10;
a compound comprising a dicarboxylic acid of a structure of formula V: 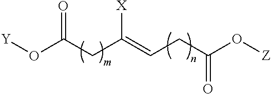
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or a C₁ to C₁₀ alkyl;
m is from 1 to 10; and
n is from 1 to 10; or
a compound comprising a dicarboxylic acid of a structure of formula VI: 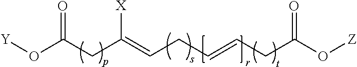
wherein X is an electron-withdrawing group selected from acyl, carboxylic acid, an ester, a halogen, fluoromethyl, —CN, sulfonyl, sulfone, sulfonic acid, primary ammonium, secondary ammonium, tertiary ammonium, or —NO₂;
Y and Z are each, independently, hydrogen or C₁ to C₁₀ alkyl;
p and t are each, independently, 1 to 10;
s is absent or 1 to 10, and
r is 1.

26-30. (canceled)

31. A method comprising mixing together (a) a liquid carrier and (b) a solid powder comprising a complex of a nitroalkene and a cyclodextrin.

32. (canceled)

33. The method of claim 31, wherein the nitroalkene is 10-nitro-octadec-9-enoic acid.

34. A method for treating a condition in a subject, comprising administering a composition of claim 1 to a subject in need thereof, wherein the condition is an inflammatory condition, an immune disease, psoriasis, obesity, metabolic syndrome, acute kidney disease, chronic kidney disease, focal segmental glomerulosclerosis, atherogenesis, adipogenesis, neointimal proliferation, kidney I/R and xenobiotic injury, focal myocardial I/R injury, Ang II-induced systemic hypertension, pulmonary hypertension, cancer, cardiac and pulmonary fibrosis, liver fibrosis, non-alcoholic steatohepatitis (NASH), non-alcoholic fatty liver disease (NAFLD), breast cancer, ovarian cancer, inflammatory bowel disease, nociception, stroke, motor neuron degeneration, diabetes, aneurysm, aortic stiffness, lupus erythematosus, STING-associated vasculopathy with onset in infancy (SAVI), asthma, chronic obstructive pulmonary disease (COPD), or focal segmental glomerulosclerosis.

35-36. (canceled)

37. A method for treating a condition in a subject, comprising administering a complex of claim 20 to a subject in need thereof, wherein the condition is an inflammatory condition, an immune disease, psoriasis, obesity, metabolic syndrome, acute kidney disease, chronic kidney disease, focal segmental glomerulosclerosis, atherogenesis, adipogenesis, neointimal proliferation, kidney I/R and xenobiotic injury, focal myocardial I/R injury, Ang II-induced systemic hypertension, pulmonary hypertension, cancer, cardiac and pulmonary fibrosis, liver fibrosis, non-alcoholic steatohepatitis (NASH), non-alcoholic fatty liver disease (NAFLD), breast cancer, ovarian cancer, inflammatory bowel disease, nociception, stroke, motor neuron degeneration, diabetes, aneurysm, aortic stiffness, lupus erythematosus, STING-associated vasculopathy with onset in infancy (SAVI), asthma, chronic obstructive pulmonary disease (COPD), or focal segmental glomerulosclerosis.
